(19) United States
(12) Patent Application Publication
(10) Pub. No.: US 2026/0237466 A1
(43) Pub. Date: Aug. 13, 2026

(54) SYSTEMS AND METHODS FOR MICROBIOME ENGINEERING

(71) Applicant: Nexilico, Inc., Danville, CA (US)

(72) Inventors: Wyatt Hartman, San Francisco, CA (US); Mohammad Soheilypour, Danville, CA (US)

(21) Appl. No.: 19/182,249

(22) Filed: Apr. 17, 2025

Related U.S. Application Data
(60) Provisional application No. 63/639,876, filed on Apr. 29, 2024.

(51) Int. Cl. G16B 40/20

(52) U.S. Cl. CPC G16B 40/20

(57) ABSTRACT

Described are systems and methods for determining one or more causal microbiome features impacting a medical condition. A method may comprise receiving, from a plurality of patients with the medical condition, a plurality of microbiome samples; generating a set of perturbed microbiomes from the plurality of microbiome samples; evaluating a set of objective functions at least in part by processing the set of perturbed microbiomes, wherein an objective function of the set of objective functions relates to the association of the microbiome with the medical condition; and identifying, by processing the set of objective functions and the set of perturbed microbiomes, the one or more causal microbiome features impacting the medical condition.

### CROSS-REFERENCE

This application claims the benefit of, and priority to U.S. Provisional Application 63/639,876, filed Apr. 29, 2024, and incorporates its disclosure herein by reference in its entirety.

### GOVERNMENT RIGHTS

This invention was made with government support under Grants No. 1938257 and No. 2228069 awarded by the National Science Foundation (NSF). The government has certain rights in the invention.

### BACKGROUND

The microbiome, e.g., the community of microorganisms that inhabit the human body and various environments, plays a key role in different fields, e.g., human health, agriculture, and industrial applications, showcasing its significance in understanding and advancing various aspects of medicine, science and technology. For human health, the microbiome has been recognized as a critical player in maintaining overall well-being, influencing digestion, immune function, and even mental health. In agriculture, the microbiome contributes to soil fertility and plant health, offering sustainable solutions for crop production. Additionally, the microbiome holds promise in industrial applications, such as biofuel production and waste treatment. The cross-disciplinary impact of microbiome research is transforming our understanding of life sciences and shaping the future of personalized medicine, agriculture, and environmental conservation.

Focusing on the human microbiome, such as the gut or skin microbiome, it emerges as a major therapeutic target with numerous programs spanning various health or medical conditions (e.g., diseases), including gastrointestinal diseases (e.g., C. difficile infection, ulcerative colitis, Crohn's disease), metabolic diseases (e.g., Type 2 Diabetes), allergic diseases (e.g., food allergy, asthma), mental disorders (e.g., hepatic encephalopathy, multiple sclerosis), autoimmune diseases, and skin diseases or conditions (e.g., psoriasis, eczema, or acne).

Considering the significance of the microbiome across application areas, substantial efforts are dedicated to engineer the microbiome for desired outcomes. However, most of these efforts have largely failed, especially for the human microbiome. The primary challenge in microbiome engineering is that interventions are primarily based on correlational biomarkers, instead of causal targets.

Identification of causal targets is particularly difficult because of the complexity and variability of the microbiome across individuals. One key aspect contributing to the complexity of the human microbiome is the diversity of microbial species and the complex web of interactions between them, numbering in the millions to billions. This complexity makes it significantly challenging to pinpoint specific microbial or molecular targets associated with particular health outcomes.

### SUMMARY

The present disclosure provides systems and methods to identify causal elements in the microbiome that could be targeted for precision engineering of the microbiome using an AI-based reverse engineering approach. The method comprises generating experimental or computational perturbation data at scale, mapping the response of target microbiomes to perturbations as training data for AI models, and using explainable AI to learn the underlying dynamics and identify causal elements that have the most positive or negative effect on the desired outcome as potential targets for a certain disease.

It should be understood that the brief description above is provided to introduce in a simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

In some example embodiments, there may be provided a method for determining one or more causal microbiome features impacting a medical condition including: receiving, from a plurality of patients with the medical condition, a plurality of microbiome samples; generating a set of perturbed microbiomes from the plurality of microbiome samples; evaluating a set of objective functions at least in part by processing the set of perturbed microbiomes, wherein an objective function of the set of objective functions relates to the association of the microbiome with the medical condition; and identifying, by processing the set of objective functions and the set of perturbed microbiomes, the one or more causal microbiome features impacting the medical condition.

In some variations, one or more of the features disclosed herein including the following features can optionally be included in any feasible combination. In some embodiments, evaluating the set of objective functions and identifying the one or more causal microbiome features are performed using at least one machine learning model. In some embodiments, the evaluating the set of objective functions is performed using a machine learning model of the at least one machine learning model and identifying the one or more causal microbiome features is performed using a post-hoc explainability process applied to the machine learning model. In some embodiments, the post-hoc explainability process comprises Local Interpretable Model-agnostic Explanations (LIME) or SHapley Additive explanations (SHAP). In some embodiments, evaluating the set of objective functions is performed using an inherently explainable machine learning model and identifying the one or more causal microbiome features is performed using feature importance or model-specific explainability methods. In some embodiments, a microbiome sample of the plurality of microbiome samples is a digitized sample collected from a patient of the plurality of patients or a synthetically generated sample. In some embodiments, a microbiome sample of the plurality of microbiome samples comprises a plurality of microbes. In some embodiments, a microbe of the plurality of microbes is a pathogen. In some embodiments, the microbe is a bacterium. In some embodiments, the method further comprises: providing the one or more causal microbiome features as input to the at least one machine learning model. In some embodiments, the medical condition is associated with a gut microbiome, a skin microbiome, a vaginal microbiome, or an oral microbiome of a patient. In some embodiments, the medical condition is a disease. In some embodiments, the disease is Crohn's disease, hepatic encephalopathy, ulcerative colitis, Type 2 diabetes, irritable bowel syndrome (IBS), Alzheimer's disease, or Parkinson's disease. In some embodiments, the medical condition is an allergy or acne. In some embodiments, the synthetically generated sample is generated using a statistical method or a generative artificial intelligence method. In some embodiments, the statistical method is Gaussian Copula. In some embodiments, the generative artificial intelligence method is a tabular variational autoencoder or a generative adversarial network (GAN). In some embodiments, generating a perturbed microbiome of the set of perturbed microbiomes comprises a modifying a metabolic landscape of a microbiome sample of the plurality of microbiome samples. In some embodiments, the modifying the metabolic landscape is performed in silico, in vitro, or in vivo. In some embodiments, the modifying the metabolic landscape relates to a modification of a microbiome composition or function. In some embodiments, the modification comprises introducing or removing one or more microbial strains, changing a concentration of metabolites, modifying an activity of a microbial gene, or any combination thereof. In some embodiments, a feature of the one or more causal microbiome features is a presence, an abundance, an activity, a concentration, or any combination thereof of a target microbiome component. In some embodiments, the target microbiome component comprises a microbe, a microbe-derived metabolite, a microbial gene, a metabolic pathway, or a combination thereof. In some embodiments, the machine learning model comprises least absolute shrinkage and selection operator (Lasso), random forests, or a neural network. In some embodiments, the inherently explainable machine learning model comprises an explainable boosting machine (EBM). In some embodiments, generating the synthetically generated sample comprises selecting a subset of nearest neighbor microbiome samples.

In some example embodiments, there may be provided a system for determining one or more causal microbiome features impacting a medical condition, comprising one or more computer processors individually or collectively programmed to: receive, from a plurality of patients with the medical condition, a plurality of microbiome samples; generate a set of perturbed microbiomes from the plurality of microbiome samples; evaluate a set of objective functions at least in part by processing the set of perturbed microbiomes, an objective function of the set of objective functions relates to the association of the microbiome with the medical condition; and identify, by processing the set of objective functions and the set of perturbed microbiomes, the one or more causal microbiome features impacting the medical condition.

In some example embodiments, there may be provided a non-transitory computer-readable medium comprising machine-executable code that, upon execution by one or more computer processors, implements a method for determining one or more causal microbiome features impacting a medical condition including: receiving, from a plurality of patients with the medical condition, a plurality of microbiome samples; generating a set of perturbed microbiomes from the plurality of microbiome samples; evaluating a set of objective functions at least in part by processing the set of perturbed microbiomes, wherein an objective function of the set of objective functions relates to the association of the microbiome with the medical condition; and identifying, by processing the set of objective functions and the set of perturbed microbiomes, the one or more causal microbiome features impacting the medical condition.

### BRIEF DESCRIPTION OF THE DRAWINGS

The present disclosure will be better understood from reading the following description of non-limiting embodiments, with reference to the attached drawings, wherein below:

FIG. 1 shows a block diagram illustrating an example module architecture for a microbiome target discovery platform for identification of causal microbiome targets for various diseases, in accordance with some embodiments;

FIG. 2 shows a block diagram illustrating an example computing system providing a computational platform for microbiome target identification for various diseases, in accordance with some embodiments;

FIG. 3 shows a block diagram illustrating an example module architecture for a microbiome digital twin platform configured to predict subject-specific microbiome response to perturbations, in accordance with some embodiments;

FIG. 4 shows a high-level flow chart illustrating an example method for processing omics data to identify microbial species and reconstruct metabolic models, in accordance with some embodiments;

FIG. 5 shows a set of graphs illustrating that in synthetic microbiomes generated based on microbiomes obtained from animal subjects, the correlation structure in microbial data is strongly preserved according to different metrics including mean taxon abundances, their covariance structures, K-L divergence, and Aitchison distance;

FIG. 6 shows a set of graphs illustrating that the microbiome digital twin platform captures the complex, multiscale dynamics of the human gut microbiota over time according to different metrics including Shannon diversity index and Aitchison distance;

FIG. 7 shows a set of graphs illustrating results for an animal study to demonstrate the dynamics of the metabolic perturbations to microbiomes are accurately captured in the microbiome digital twin platform according to different methods including Principal Coordinate Analysis (PCoA) of microbial compositions and Aitchison distance;

FIG. 8 shows a graph illustrating highly reproducible results of perturbation experiments using the microbiome digital twin platform;

FIG. 9 shows a set of graphs illustrating that various artificial intelligence (AI) models could accurately predict perturbation outcomes using the data generated by the microbiome digital twin platform using observed samples;

FIG. 10 shows a set of graphs illustrating that the use of synthetic samples could result in development of more generalizable AI models;

FIG. 11 shows a set of graphs illustrating an explainable AI method that can identify potential microbiome engineering targets by identifying top microbiome components that have a positive or negative impact on the desired engineering outcome using observed samples or combination of observed and synthetic samples; and

FIG. 12 shows a set of graphs illustrating another explainable AI method that can identify potential microbiome engineering targets by identifying top microbiome components and their interactions that have a positive or negative impact on the desired engineering outcome using observed samples or combination of observed and synthetic samples.

### DETAILED DESCRIPTION

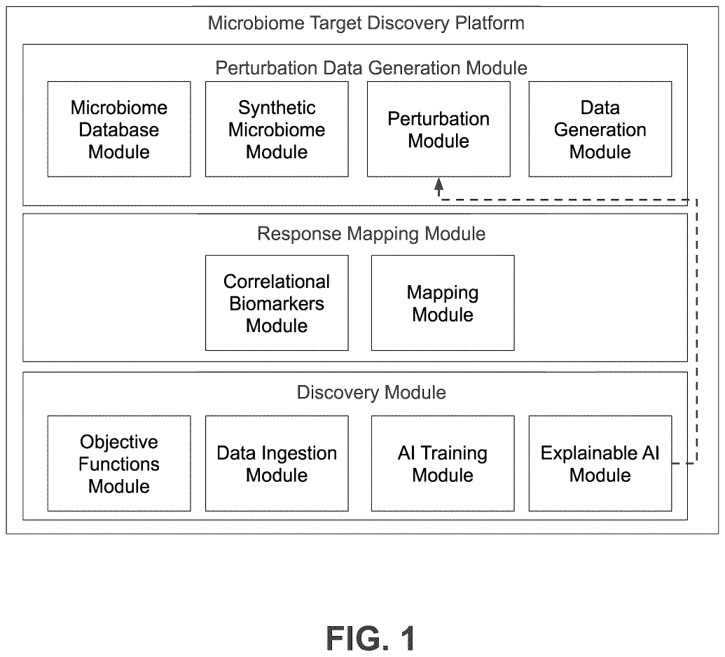

FIG. 1 shows a block diagram illustrating an example module architecture for a microbiome target discovery platform 100 for identification of causal microbiome targets for various health or medical conditions (e.g., diseases). It should be appreciated that the modules of the target discovery platform 100 are exemplary and non-limiting, and that the target discovery platform 100 may be implemented with other modules and sub-modules without departing from the scope of the present disclosure. A microbiome as described herein can be, for example, a gut microbiome, a skin microbiome, a vaginal microbiome, or an oral microbiome.

The target discovery platform 100 comprises a plurality of modules, including a perturbation data generation module 110 configured to generate microbiome perturbation data for a certain disease at scale, a response mapping module 120 configured to map microbiome response to perturbations according to microbiome biomarkers, and a discovery module 130 configured to identify causal microbiome targets using artificial intelligence and explainable methods. A disease can be, for example, Crohn's disease, hepatic encephalopathy, ulcerative colitis, Type 2 diabetes, irritable bowel syndrome (IBS), Alzheimer's disease, or Parkinson's disease. Other medical or health conditions associated with the systems and methods described herein can include allergies or acne.

The perturbation data generation module 110 may comprise a microbiome database module 112 configured to store omics data, including one or more of metagenomic, metatranscriptomic, metaproteomic, or metabolomic data, from a set of microbiome samples from patients with a certain disease. As an illustrative and non-limiting example, the microbiome database module could include tens of metagenomic samples from patients with a certain disease to capture a wide microbiome inter-individual variability.

The perturbation data generation module 110 may further comprise a synthetic microbiome module 114 configured to generate synthetic microbiome profiles based on the microbiome samples stored in the microbiome database module 112. Since the number of perturbations may be larger than that of the subjects, synthetic microbiome backgrounds may be used to avoid potential overfitting of the artificial intelligence models to a small number of subject backgrounds. Synthetic backgrounds could be generated using statistical methods or artificial intelligence models. As illustrative and non-limiting examples, generative methods such as Gaussian Copula, tabular variational autoencoders, or generative adversarial networks (GANs) such as Copula GAN, conditional table GAN, and Wasserstein GAN may be used.

As an illustrative and non-limiting example, synthetic microbiomes may be generated using Gaussian Copula by simulating, e.g., at least 10,000, at least 100,000, or at least 1 million synthetic samples based on 25 initial subject microbiomes. To select more realistic synthetic microbiomes, 4 nearest neighbor samples of each of the 25 initial subjects may be chosen from the pool of synthetic microbiome samples. Synthetic data quality may be assessed by comparing mean taxon abundances of synthetic samples and their covariance structures, along with similarity metrics (e.g., K-L divergence or Aitchison distance). In some implementations, alternative-size subsets of nearest neighbor samples are chosen.

The perturbation data generation module 110 may further comprise a perturbation module 116 configured to set up a set of metabolic perturbations to be applied to microbiome samples from patients with a certain disease. The perturbations may be performed by any modification in the metabolic landscape of the microbiome to modify microbiome composition and function, including but not limited to adding or removing one or more microbial strains, changing the concentration of metabolites, modifying the activity of microbial genes, or any combination thereof.

As an illustrative and non-limiting example, an in house or publicly available database of microbial strains may be used to generate a list of combinations of microorganisms that could be introduced to target microbiomes to invoke a range of metabolic perturbations. A microbe described herein can be a pathogen (e.g., a bacterium). For example, the Unified Human Gastrointestinal Genome (UHGG) collection, PubSEED, Human Gastrointestinal Bacteria Culture Collection, or AGORA2 (assembly of gut organisms through reconstruction and analysis, version 2) could be used as reference databases. The combinations of microorganisms could be selected randomly, according to distributions across microbial strains, species, genera, families, orders, classes, or phyla or specific to a set of microbial strains, species, genera, families, orders, classes, or phyla of interest for a certain disease.

Perturbations may be selected randomly or be designed according to prior knowledge about the disease. Additional perturbations may be designed based on the observations, data, and outcomes of the artificial intelligence module 134 and the explainable AI module 136, according to a feedback loop.

The perturbation data generation module 110 may further comprise a data generation module 118 configured to generate the perturbation data for target discovery. Microbiome perturbations could be performed in silico, in vitro, or in vivo. However, it should be noted that perturbations should be done at a large scale, e.g. tens, hundreds, thousands, or millions of microbiome perturbations per target disease. As an illustrative and non-limiting example, microbiomes could be perturbed using the microbiome digital twin platform 300. It should be appreciated that any computational or experimental approach or method or any combination thereof may be used without departing from the scope of the present disclosure.

The response mapping module 120 may comprise a correlational biomarkers module 122 configured to store, identify, or characterize microbiome biomarkers associated with target patient population. As illustrative and non-limiting examples, correlational biomarkers, including but not limited to taxonomic or metabolic biomarkers, may be previously known or associated with any combination of data associated with a microbiome, such as relative abundance of microbial species, concentration of microbiome-derived metabolites, microbial genes, or activity of metabolic pathways, as illustrative and non-limiting examples.

In the case of unknown microbiome biomarkers, they may be identified, as an illustrative and non-limiting example, by first collecting and/or retrieving fecal samples from patients with the target indication as well as healthy control individuals and then characterizing key taxonomic, genetic, transcriptomic, proteomic, or metabolic differences between the two cohorts using metagenomic, metatranscriptomic, metaproteomic, metabolomic sequencing or any combination thereof.

It should be appreciated that correlational biomarkers could be already known and obtained from available data and literature. Additionally, metagenomic, metatranscriptomic, metaproteomic, metabolomic data or any combination thereof could already be accessible through private or public databases.

Correlational biomarkers may be identified by a variety of methods including but not limited to comparing the abundance of microorganisms at phylum, class, order, family, genus, species, or strain level in healthy individuals versus patients and identify phyla, classes, orders, families, genera, species, or strains that are statistically significantly higher or lower in abundance in patients over healthy individuals, comparing concentration of metabolites or classes of metabolites in healthy individuals versus patients to identify metabolites that are statistically significantly higher or lower in concentration in patients over healthy individuals, using bioinformatics and/or machine learning methods to identify microbiome biomarkers, including but not limited to microorganisms at any taxonomic level, metabolite concentrations, diversity metrics such as alpha or beta diversity, microbial gene content, microbial gene products, microbiome-derived metabolites, or microbial pathways or any combination thereof that are significantly correlated with endpoints of interest for the target indication.

It should be appreciated that host features and/or clinical endpoints may be used in combination and/or instead of microbiome biomarkers without departing from the scope of the present disclosure. As an illustrative and non-limiting example, area under the curve (AUC) of core body temperature may be used as a clinical endpoint. As another example, a host's metabolic and/or immune profile may be used as a host feature. As another example, plasma levels of certain metabolites could be used as a clinical endpoint. Host features may be characterized using genomics, transcriptomics, proteomics, metabolomics, or any combination thereof.

The response mapping module 120 may further comprise a mapping module 124 configured to calculate and/or measure the response of the target microbiomes to perturbations according to the correlational biomarkers identified in the correlational biomarker module 122. The calculations and/or measurements could be at the molecular and/or microbial level or any combination thereof. The response includes the evaluation of correlational biomarkers in each perturbation experiment. As an example, higher abundance of specific species from bacterial genera such as Faecalibacterium, Akkermansia, Clostridia, Lactobacillus, Bifidobacteria, some Bacteroides, and Alistipes, as illustrative and non-limiting examples, (Group I) may be positively associated with a disease, while higher abundance of some specific species from bacterial genera such as Enterobacteria and Bacteroides, as illustrative and non-limiting examples (Group II), may be negatively associated with a certain disease. In that case, individual and total relative abundance of these microbial species will be measured or calculated in each experiment or simulation.

It should be appreciated that methods to calculate or measure microbiome response may be based on the methods employed in the data generation module 118 to conduct the perturbation experiments including in silico, in vitro, or in vivo approaches. As an illustrative and non-limiting example, in an in silico approach, parameters associated with abundance of microbial species could be tracked during each simulation and relative abundance of target species could be calculated at the end of each simulation. As another illustrative and non-limiting example, in a high-throughput in vitro approach, concentration of a certain molecule could be tracked using fluorescent imaging techniques. The outcome of this step may be an atlas of microbiome response based on correlational biomarkers to a variety of perturbations.

The discovery module 130 may comprise an objective functions module 132 configured to convert correlational microbial biomarkers to mathematical equations that may be used for training and validation of one or more artificial intelligent models. As an example, higher abundance of specific species from bacterial genera such as Faecalibacterium, Akkermansia, Clostridia, Lactobacillus, Bifidobacteria, some Bacteroides, and Alistipes, as illustrative and non-limiting examples (Group I) may be positively associated with a disease, while abundance of some specific species from bacterial genera such as Enterobacteria and Bacteroides, as illustrative and non-limiting examples (Group II) may be negatively associated with a certain disease.

Therefore, a higher abundance of Group I and a lower burden of Group II may be desired. Therefore, the loss functions for training of one or more artificial intelligence models may be mean absolute error (MAE) for (1) relative abundance of Group I bacterial species, and (2) relative abundance of Group II bacterial species. Sₜₒₜ may be the sum of relative abundances of species in Group I, [symbol] the sum of predicted relative abundances of these species, Fₜₒₜ may be the sum of relative abundances of species in Group II, and [symbol] the sum of predicted relative abundances of these species. Therefore, Group I and Group II loss functions may be defined according to:

$${S}_{loss} = \frac{1}{n} {\sum }_{i = 1}^{n} \vert \text{?} - {S}_{tot} \vert$$

$${F}_{loss} = \frac{1}{n} {\sum }_{i = 1}^{n} \vert \text{?} - {F}_{tot} \vert$$

The discovery module 130 may further comprise a data ingestion module 134 configured to prepare the input data for training of artificial intelligence models. As illustrative and non-limiting examples, taxonomic IDs, microbial genomic data, metabolomic data, metabolic networks or any combination thereof may be ingested from the atlas of microbiome response to perturbations as input data. It should be appreciated that input data may be used in any form without departing from the scope of the present disclosure.

The discovery module 130 may further comprise an AI Training module 136 configured to train and validate one or more artificial intelligence models using the atlas of microbiome response to perturbations from the mapping module 124. The input to these models may be the data from the data ingestion module 134 and the composition of microbiome samples used in data generation module 118 and the output may be objective functions outlined in the objective functions module 132.

As an illustrative and non-limiting example, the artificial intelligence model may comprise a machine learning or a deep learning model such as least absolute shrinkage and selection operator (Lasso), Random Forest (RF), Explainable Boosting Machine (EBM), or fully connected neural network. An 80-20 split in the training dataset may be used. All the inputs and outputs may be normalized from zero to one to ensure quicker convergence. Therefore, relative abundance for all the microbial strains may be used. The minimum and maximum will be calculated based on the training set and those values will be used to normalize both the training and testing set. For deep learning models, rectified linear units (ReLUs) can be used for activation functions, and dropout layers may be placed directly after any fully connected layers to prevent overfitting. Dropout layers may be at uniform intervals between 0.1 and 0.5. The Adam optimizer may be used to speed up the convergence of the models.

The discovery module 130 may further comprise an explainable AI module 138 configured to identify components of the microbiome that may have a positive or negative effect on the desired outcome as potential targets. Desired outcome is determined according to the objective functions outlined in the objective functions module 132. As an illustrative and non-limiting example, Shapley Additive Explanation (SHAP) or Local Interpretable Model-agnostic Explanations (LIME) may be used to provide explainability for the AI models. It should be appreciated that any other post-hoc explainability approach or method may be used without departing from the scope of the present disclosure. Additionally, use of inherently explainable machine learning models and using feature importance or other model-specific explainability approach or method will not depart from the scope of the present disclosure. Potential targets could be any component of a microbiome. As illustrative and non-limiting examples, microbes, microbe-derived metabolites, microbial genes, metabolic pathways, or any combination thereof could be identified as potential targets.

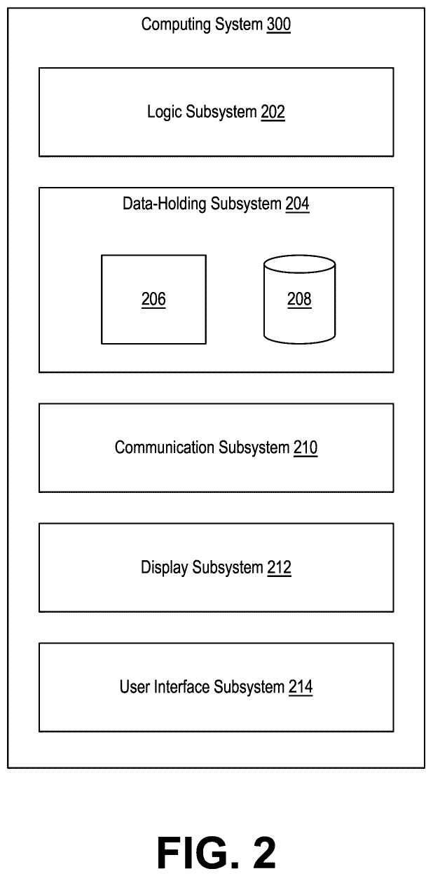

Different modules of the microbiome target discovery platform 100 may be implemented in a computing system. FIG. 2 shows a block diagram illustrating an example computing system 200 providing a computational platform for microbiome target identification for various diseases. It should be appreciated that the architecture of the computing system 200 is exemplary and non-limiting, and that other computer architectures may be used for a computing device without departing from the scope of the present disclosure. It should also be appreciated that the data generation module 118 would only be part of the platform implemented in computing system 200 if in silico methods are used.

In different embodiments, the computing system 200 may comprise a mainframe computer, a server computer, a desktop computer, a laptop computer, a tablet computer, a network computing device, a mobile computing device, a mobile communication device, and so on. As depicted, the computing system 200 comprises a logic subsystem 202 and a data-holding subsystem 204. The computing system 200 may further include a communication subsystem 210, a display subsystem 212, and a user interface subsystem 214.

The logic subsystem 202 may include one or more physical devices configured to execute one or more instructions. For example, the logic subsystem 202 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem 202 may include one or more processors that are configured to execute software instructions. In some examples, the logic subsystem 202 may include one or more hardware and/or firmware logic machines configured to execute hardware and/or firmware instructions. Processors of the logic subsystem 202 may be single core or multi-core, and the programs executed thereon may be configured for parallel or distributed processing. The logic subsystem 202 may optionally include individual components that are distributed throughout two or more devices, which may be remotely located and/or configured for coordinated processing. One or more aspects of the logic subsystem 202 may be virtualized and executed by remotely accessible networked computing devices configured in a cloud computing configuration.

The data-holding subsystem 204 may include one or more physical, non-transitory devices configured to hold data and/or instructions executable by the logic subsystem 202 to implement the herein-described methods and processes. When such methods and processes are implemented, the state of data-holding subsystem may be transformed (for example, to hold different data).

The data-holding subsystem 204 may include removable media and/or built-in devices. Data-holding subsystem 204 may include optical memory (for example, CD, DVD, HD-DVD, Blu-Ray Disc, and so on), and/or magnetic memory devices (for example, hard disk drive, floppy disk drive, tape drive, and magnetoresistive random access memory (MRAM)), and the like. The data-holding subsystem 204 may include devices with one or more of the following characteristics: volatile, nonvolatile, dynamic, static, read/write, read-only, random access, sequential access, location addressable, file addressable, and content addressable. In some embodiments, the logic subsystem 202 and the data-holding subsystem 204 may be integrated into one or more common devices, such as an application specific integrated circuit (ASIC) or a system on a chip (SoC). In other embodiments, the data-holding subsystem 204 may include individual components that are distributed throughout two or more devices, which may be remotely located and accessible through a networked configuration.

When included, the communication subsystem 210 may be configured to communicatively couple the computing system 200 with one or more other computing devices. The communication subsystem 210 may include wired and/or wireless communication devices compatible with one or more different communication protocols. As non-limiting examples, the communication subsystem 210 may be configured for communication via a wireless telephone network, a wireless local area network, a wired local area network, a wireless wide area network, a wired wide area network, and so on. In some examples, the communications subsystem 210 may enable the computing system 200 to send and/or receive messages to and/or from other computing systems via a network such as the public Internet.

When included, the display subsystem 212 may be used to present a visual representation of data held by data-holding subsystem 204. As the herein-described methods and processes change the data held by the data-holding subsystem 204, and thus transform the state of the data-holding subsystem 204, the state of display subsystem 212 may likewise be transformed to visually represent changes in the underlying data. The display subsystem 212 may include one or more display devices utilizing any type of display technology. Such display devices may be combined with the logic subsystem 202 and/or the data-holding subsystem 204 in a shared enclosure, or such display devices may comprise peripheral display devices.

When included, the user interface subsystem 214 may include one or more physical devices configured to facilitate interactions between a user and the computing system 200. For example, the user interface subsystem 214 may comprise one or more user input devices including but not limited to a keyboard, a mouse, a camera, a microphone, a touch screen, and so on.

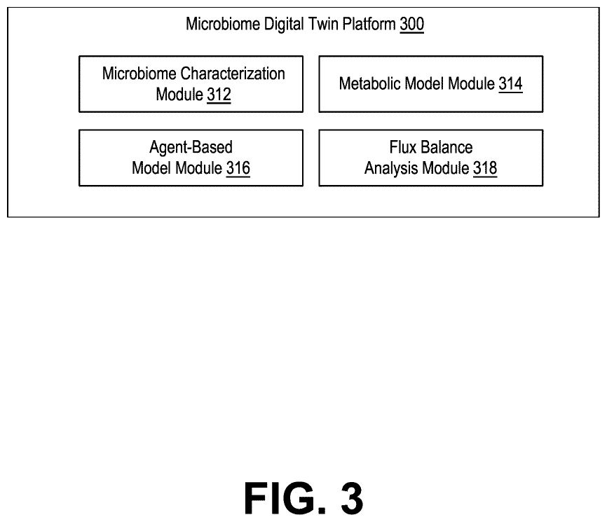

FIG. 3 shows a block diagram illustrating an example module architecture for a microbiome digital twin platform 300 configured to predict subject-specific microbiome response to perturbations. It should be appreciated that the modules of the microbiome digital twin platform 300 are exemplary and non-limiting, and that the microbiome digital twin platform 300 may be implemented with other modules and sub-modules without departing from the scope of the present disclosure.

The microbiome digital twin platform 300 comprises a plurality of modules, including a microbiome characterization module 302, a metabolic model module 304, an agent-based model module 306, and a flux balance analysis module 308. The microbiome characterization module 302 may extract types and abundance of microbial species from omics data.

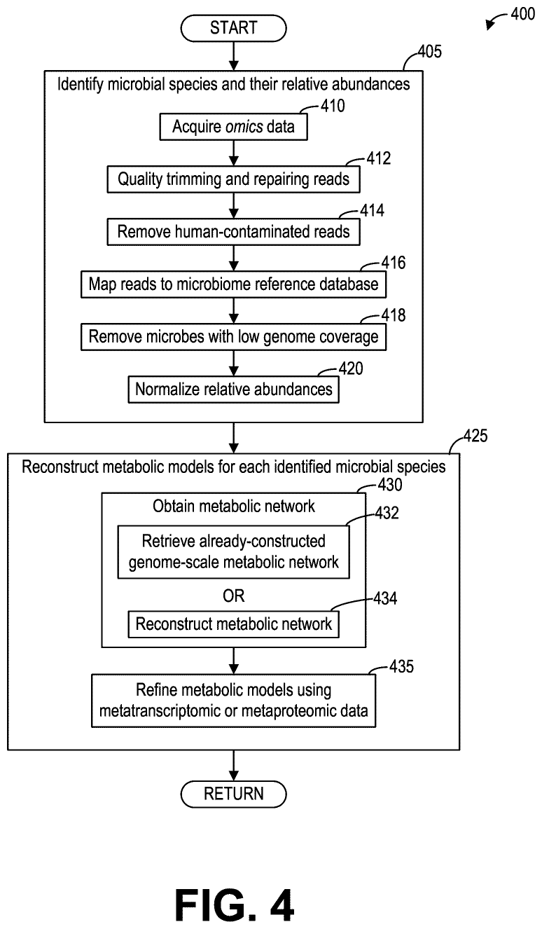

For example, FIG. 4 shows an illustrative and non-limiting example method 400 for the microbiome characterization module 302. Method 400 begins at 405, where method 400 identifies microbial species and their relative abundances. To that end, method 400 obtains raw 16S ribosomal RNA (rRNA) or metagenomic data either through 16S rRNA or shotgun sequencing of the target microbiome or via the National Center for Biotechnology Information (NCBI) sequence read archive (SRA) or any similar database. It should also be appreciated that microbiome profiling may be performed using other omics data or a combination thereof.

At 412, method 400 may quality trim the reads using Trimmomatic and then may re-pair the reads using the BBmap repair tool. At 414, method 400 may remove human contaminant sequences by mapping the paired reads to human reference genome such as build 38 (GRCh38) using Burrows-Wheeler Aligner (BWA). Cross-mapped reads (reads mapped to multiple positions) may be filtered out by discarding mapped reads with a low-quality score using SAMtools. At 416, method 400 then may map the pre-processed reads to a reference gut microbiome database.

At 418, method 400 may remove microbes with low genome coverage. For example, the abundance of each microbial species may be calculated by adding up the sequence length of reads mapped to a unique region of a species' genome, normalized by the total size of the species' genome. A minimum genome coverage (for example, 1%) may be assigned for each identified microorganism to reduce the number of false positives. At 420, the resulting coverages for each microorganism may be normalized to 1 Gb to obtain relative microbe abundances.

After identifying the microbial species and their relative abundances at 405, method 400 proceeds to 425, where method 400 reconstructs metabolic models for each identified microbial species. Genome-scale metabolic models relate metabolic genes with metabolic pathways. Thus, at 430, the metabolic model module 314 can retrieve or reconstruct the metabolic models associated with the microorganisms identified by microbiome characterization module 312.

The metabolic model module 312 may use metabolic model datasets at 432, in some examples, or in other examples the metabolic model module 312 may, at 434, use metabolic network reconstruction methods or tools, such as the CarveMe tool, to build metabolic models using reference genomes. After obtaining the metabolic networks, method 400 continues to 435.

At 435, method 400 may further refine the metabolic models using metatranscriptomic, metaproteomic, or metabolomic data. First, gene or protein expression data is binarized into on and off states. Subsequently, these states are used to modify metabolic pathways by mapping to corresponding genome-scale metabolic network reconstructions. The reconstructed metabolic models may be associated with a corresponding agent type in the agent-based model(s) module 316.

Referring again to FIG. 3, the agent-based model(s) module 316 constructs a subject-specific model of a target microbiome. The primary inputs to the model may be microbial species identified in microbiome characterization module 312, relative abundance of each microorganism identified in microbiome characterization module 312, metabolic networks associated with each microorganism identified in metabolic model(s) module 314, and metabolites that should be present to support these metabolic pathways.

Metabolic perturbations may be introduced in different forms. As an illustrative and non-limiting example, gut-associated microorganisms could be added or removed to invoke a metabolic perturbation. In that case, those microbes may be added to the system similar to host bacteria and their metabolic models are integrated with the rest of the bacteria in the system. As another illustrative and non-limiting example, metabolite concentrations may be altered or metabolites may be added or removed to invoke a metabolic perturbation.

Additional inputs to the model may include simulation parameters such as the size of the system (e.g., in micrometers), the time step (e.g., in seconds), and the number of desired simulation steps as well as molecular fields in the system, their diffusion coefficients, and their initial concentrations. The agent-based model(s) module 316 may then construct the three-dimensional environment of the simulation where agents (representing microorganisms) are distributed randomly, with each microbe given random initial biomass according to a median cell dry weight (e.g. 0.489 μg) and a dry weight deviation (e.g. 0.132 μg).

The modeling environment in agent-based model(s) module 316 may be discretized at the molecular scale and the initial concentration of molecular fields may be assigned to each grid cell. Molecular species (e.g., metabolites) may be modeled using ordinary differential equations (ODEs) and allowed to diffuse between boxes with the diffusion of molecules governed by Fick's Second Law:

$$\frac{\partial [ C ]}{\partial T} = D ( \frac{{\partial }^{2} [ C ]}{\partial {x}^{2}} + \frac{{\partial }^{2} [ C ]}{\partial {y}^{2}} + \frac{{\partial }^{2} [ C ]}{\partial {z}^{2}} )$$

Diffusion may be modeled using the algorithm proposed by Grajdeanu. Based on this algorithm, the concentration in each grid cell can depend on the concentration in neighboring grid cells, the distance between cells, and the diffusion coefficient, which may be calculated according to:

$${d}_{j} = \vert {x}_{i} - {x}_{i}^{j} \vert$$

$$C ( {x}_{i} ) = C ( {x}_{i} ) + A {\sum }_{j = 1}^{n} ( C ( {x}_{i}^{j} ) - C ( {x}_{i} ) ) {e}^{- {d}_{j}^{2} / D}$$

$$A {\sum }_{j = 1}^{n} {e}^{- {d}_{j}^{2} / D} = 1$$

The movement of agents (representing microbes) may be modeled by random walk (suggested for time steps greater than 30 minutes) or biophysical flagellar movement, such as running and tumbling. A pairwise collision force may be applied to all overlapping microorganisms to avoid collision of diffusing bacterial agents. The magnitude of this force is proportional to the log of the ratio of the distance between two bacteria centers and the sum of their radii.

The agent-based model(s) module 316 may then run the simulation, also referred to herein as the in silico experiment, for the desired number of time steps. At each time step, a range of data may be stored such as coordinates of microorganisms, cell population, and the concentration of molecular fields. Microorganisms may be represented by autonomous agents possessing cellular characteristics including growth, division, and migration.

Microorganism growth, death, and division rules and rates may be naturally calculated from metabolic interactions or implemented based on experimental studies of morphogenesis in individual bacteria. In addition to characteristics of agents, agent-based model tools may provide other aspects of the simulation such as environmental boundaries, physical factors (e.g., crowding and steric repulsion), and collision detection.

The flux balance analysis module 318 uses flux balance analysis to predict metabolic interactions of microorganisms with the environment, and hence, identify their microbial growth. The flux balance analysis module 318 calculates the flow of metabolites through biochemical reactions in a metabolic network using any form of flux balance analysis, e.g. individual flux balance analysis or community flux balance analysis. The fluxes may be computed by optimizing an objective function

$$Z = {c}^{T} v$$

where v is the vector of target fluxes. The linear programming problem can solve:

$$S \cdot v = 0$$

where S is an m×n stoichiometric matrix of biochemical reactions with m compounds and n reactions, subject to lower and upper bounds for the vector v and a linear combination of fluxes Z as the objective function. Each agent may be assigned its metabolic models according to agent type. A linear programming (LP) solver such as GLPK (GNU Linear Programming Kit) or COIN-OR Linear Programming (CLP) may be used to solve LP problems for flux balance analysis. Lower bounds of fluxes may be updated according to the local concentration of metabolites in the vicinity of the microorganism. At each time step, LP solver may solve LP problems for each microorganism and update environmental concentrations of the metabolites that are involved in exchange metabolic interactions.

Additionally, the biomass accumulated by an individual agent may be updated according to an exponential growth model using the optimal biomass flux computed by FBA

$${Biomass}_{t + 1} = {Biomass}_{t} + {v}_{biomass} \times {Biomass}_{t} \times dt$$

Once accumulated biomass reaches a maximal dry weight (e.g. 1.172 μg), microbes replicate. When the accumulated biomass drops below a minimal dry weight (e.g. 0.083 μg), microorganisms die.

At each time step, molecular fields may be evaluated and field concentrations may be updated according to metabolic interactions of microorganisms.

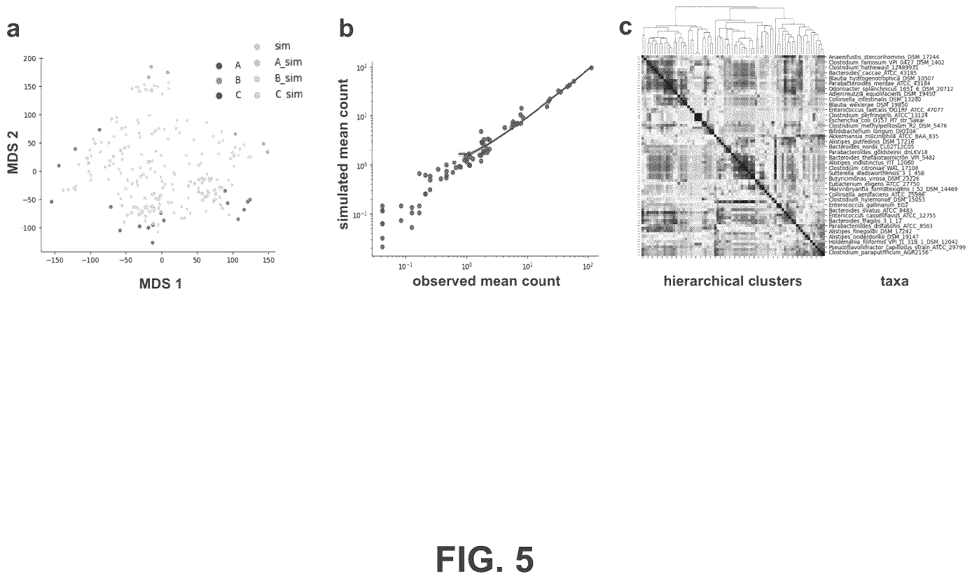

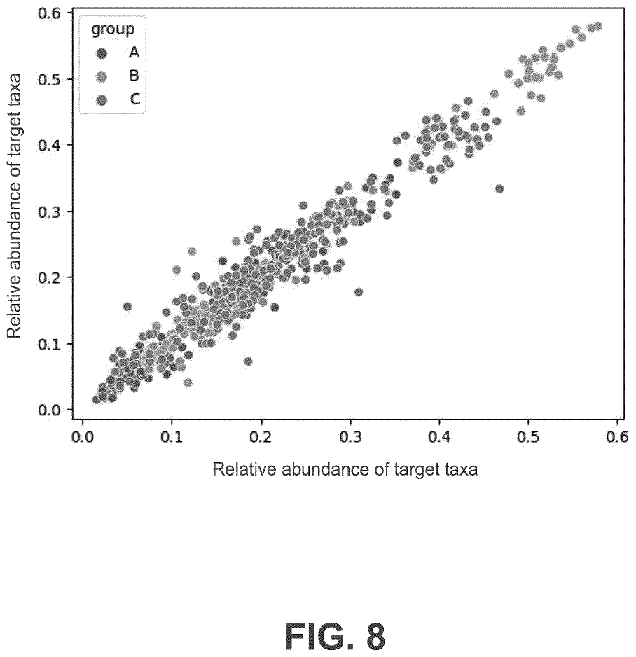

To demonstrate the accuracy and advantages of the systems and methods provided herein relative to previous approaches, the results of multiple modeling and analysis studies are illustrated in FIGS. 5-12.

FIG. 5 depicts a set of graphs illustrating results of using Gaussian Copula methods to generate synthetic microbiomes based on 25 microbiomes obtained from animal subjects humanized by three different fecal samples. The three panels demonstrate comparison of synthetic microbiome data to observed animal subject data. Synthetic data was generated by simulating 100,000 synthetic samples based on 25 initial subject microbiomes. In panel (a), to select more realistic synthetic microbiomes, 4 nearest neighbor samples of each of the 25 initial subjects were chosen from the pool of synthetic samples. Panels (b) and (c) demonstrate mean taxon abundances of synthetic data and their covariance structures, respectively to assess synthetic data quality. Other similarity metrics, e.g., K-L divergence and Aitchison distance, may be used as well. This comparison demonstrated that the correlation structure in microbial data is strongly preserved.

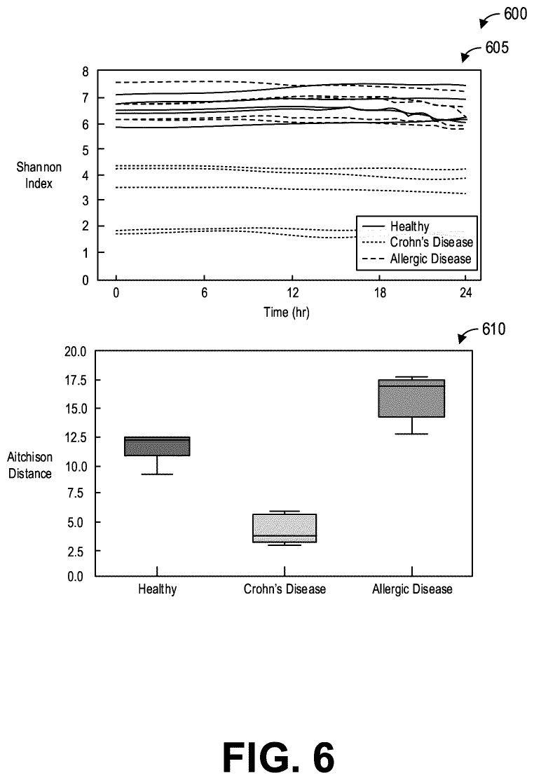

The accuracy of the microbiome digital twin platform 300 in modeling and simulation of subject-specific microbiomes and their response to perturbations is demonstrated in multiple studies. FIG. 6 depicts a set of graphs 600 illustrating results for twenty-four hours in silico experiments using the microbiome digital twin platform 300 for fifteen human samples, including 10 samples from a study of early onset Crohn's disease (CD) patients and 5 samples from a cohort of individuals with allergic diseases who participated in a Phase I clinical trial. For the CD study, paired-end Illumina® raw reads for five healthy controls and five CD patients were retrieved from NCBI SRA under the accession SRP057027. For allergic patients, paired-end Illumina® raw reads were provided by Siolta Therapeutics, Inc. Each microbiome was constructed using the microbiome digital twin platform 300 and simulated for 24 hours with a time step of one hour. Alpha diversity and Aitchison distance were monitored throughout the simulation. Alpha diversity was calculated using the Shannon diversity index and is depicted in the graph 605. Aitchison distance was calculated by taking the Euclidean distance between the centered-log transformed samples, and is depicted in the graph 610. For each simulated microbiome sample, it is expected that, in the absence of external stimuli, the composition of the microbiome over the time of simulation would not deviate from its initial composition. FIG. 6 depicts that all the microbiome samples show a change of <10% in Shannon index throughout the simulation and an Aitchison distance of <20 between the final and initial composition of the simulated microbiome, confirming that the complex, multiscale dynamics of the human gut microbiota is captured over time.

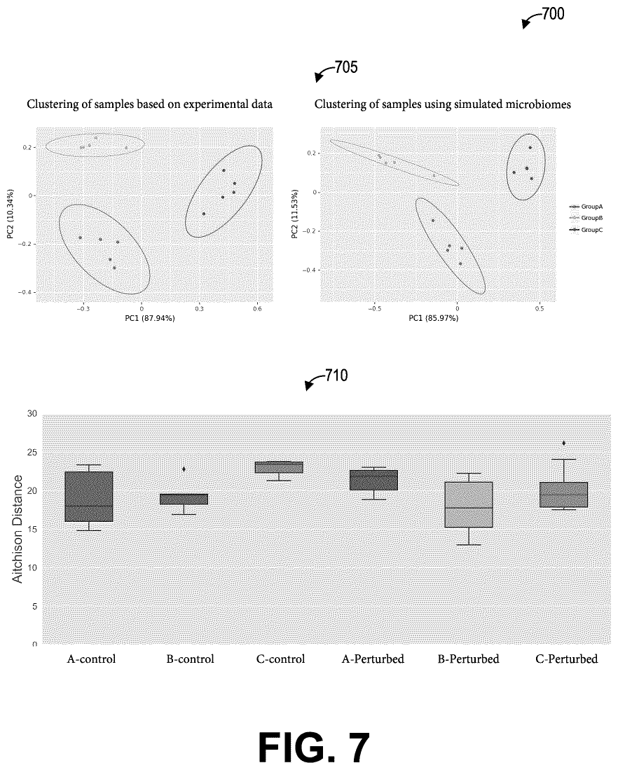

FIG. 7 depicts a set of graphs 700 illustrating results for an animal study to demonstrate the dynamics of the metabolic interactions between microorganisms in a microbiome are accurately captured in the microbiome digital twin platform 300. Taxonomic data obtained from mice fecal samples were used to build subject-specific models of the gut microbiome for each mouse in this group (total of 5 for each group). Each sample was simulated for 7 days with a time step of 1 hour (total of 168 hours) to replicate the experiments.

Graph 705 depicts that, using Principal Coordinate Analysis (PCoA), it could be seen that mice from each microbiota background will cluster together when the system uses taxonomic data obtained from fecal samples after 7 days. Similarly, simulated compositions (final composition after a 7-day simulation) using the microbiome digital twin platform 300 also resulted in similar clustering of mice from different microbiota backgrounds.

Graph 710 shows the compositional difference between experimental and simulated microbiome compositions using Aitchison distance across three groups of mice (A, B, and C) with distinct baseline microbiota compositions. An Aitchison distance of <25 is representative of closely related microbiome compositions between two samples. Graph 710 shows the Aitchison distance for all the 15 simulated samples from control groups (absence of external stimuli, e.g. a live biotherapeutic product) is <25, demonstrating that the simulated control microbiomes are closely related in composition to the experimental data. These comparisons confirm that the platform accurately captures the time-dependent characteristics of the community of microorganisms in the gut microbiome in the absence of external stimuli.

Graph 710 further shows the compositional difference between experimental and simulated microbiome compositions perturbed by external microbial strains using Aitchison distance. Again, taxonomic data obtained from mouse fecal samples were used to build subject-specific models of the gut microbiome for each mouse in the supplemented group (total of 25 mice). The initial relative abundance of external strains was calibrated with a trial-and-error process. Each sample was simulated for 7 days with a time step of 1 hour (total of 168 hours) to replicate the experiments.

Graph 710 shows the Aitchison distance for all the 25 simulated samples from the supplemented group is <25, confirming that the composition of simulated microbiomes in the presence of perturbation is significantly similar to experimentally-obtained microbiome compositions.

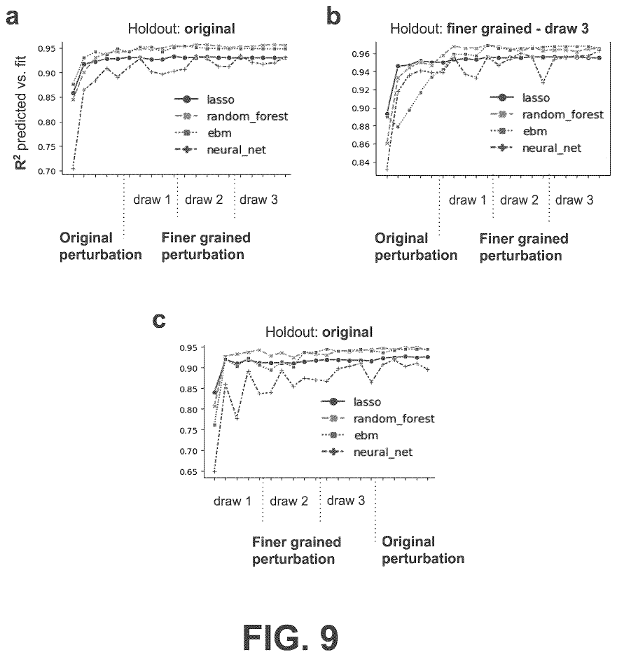

The microbiome digital twin platform 300 was further evaluated to ensure result reproducibility across experiment replicates of perturbations. A total of 25 animal subjects were used to build a total of 725 simulations using the microbiome digital twin platform 300 by 29 perturbations per sample according to the perturbation module 116. FIG. 9 shows reproducibility results of relative abundance of correlational biomarkers obtained from 725 simulations of perturbations of microbiomes from 25 mice humanized by 3 different fecal samples. The plot shows that the microbiome digital twin platform achieves a reproducibility index of R2 of >0.94.

The AI training module 136 was evaluated to demonstrate the capability of various AI models to accurately predict desired outcomes according to the objective functions module 132. The perturbation module 116 was used to generate 725 perturbation experiments, called the “original perturbation set”, by designing 29 perturbations for each of the 25 animal subjects. A collection of 232,000 additional finer grained perturbation experiments were generated using the perturbation module 116, from which 1,800 random experiments were obtained using 3 random draws of 600 perturbations each, with 100 held out per draw.

FIG. 9 shows the change in fit (R2) of predicted objective functions for 100 holdout data observations versus model predicted for the 25 animal subjects. Model fits change while more training data is added sequentially, with each label representing 100 new samples. Models compared included Lasso, Random Forest (RF), Explainable Boosting Machine (EBM), and a fully connected neural network (NN). Panel (a) shows change in model predicted fit (R2) of the original perturbation set holdout samples, as original perturbation set batches of 100, then three draws of finer grained perturbation data were added to model training. The models reach R2 of >0.95 within a few hundred samples for original perturbation data when the model was trained on the same perturbation data distribution. Random forest (RF) and explainable boosting machine (EBM) models fit best with least data, while a simple neural network (NN) model continued to improve fit when exposed to finer grained perturbations.

Panel (b) shows change in model predicted fit (R2) of finer grained perturbation data held out from the third draw, with accumulation of original perturbation set and finer grained perturbation training data. Prediction of these finer grained perturbation data from original perturbation data did not reach >0.95 without exposure to other finer grained perturbation data for more complex models (RF, EBM, NN), which did not improve fit of simpler Lasso models.

Panel (c) shows change in model predicted fit (R2) of original perturbation set holdout samples as finer grained perturbation data draws, then original perturbation data were added to the training data. When original perturbation data holdout data was predicted using models trained on finer grained distributed data, a few hundred samples were sufficient for training of simpler Lasso and RF models. Additional training data improved prediction using EBM and NN models.

Results in FIG. 9 illustrate the potential for more complex models (EBM and NN) to overfit to limited training data or require more data for adequate training, as described by the bias-variance tradeoff. Importantly, these results also show the potential for cross training of more complex models to learn to predict different perturbation datasets. Such cross training may make more generalizable models with less sample bias. This generalizability is partly illustrated by good fits to random samples obtained from a large number (232,000) of finer grained perturbations, as illustrated in panel (c).

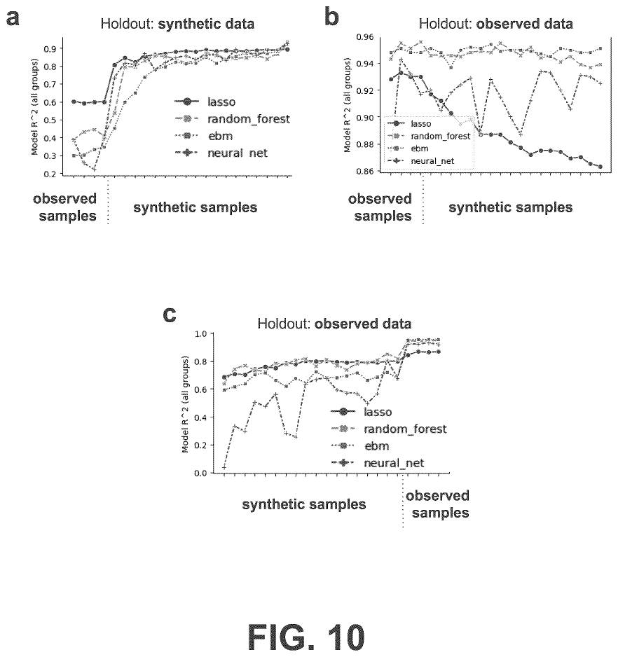

However, each of the results in FIG. 9 were obtained using only 25 animal subject microbial backgrounds. Bias might arise due to models overfitting to specific animal subject backgrounds, given the number of perturbations is overwhelmingly larger than that of animal subjects. Accordingly, the system generated synthetic microbiome samples based on the observed subjects. FIG. 10 shows model holdout performance for synthetic and observed microbiome samples, spiked with different combinations of perturbation data from observed and synthetic samples. Each experiment label represents 500 experiments in the training set (13,925 total). Synthetic data was drawn from nearest neighbors of observed data from sampling distributions of 10,000, 100,000, and 1 million points. Three different draws were taken from each distribution (D1-D3) to generate synthetic microbiome backgrounds perturbed with similar perturbations as the observed subject microbiome backgrounds.

Panel (a) shows that training on only observed subject data generates models that are very poorly predictive of synthetic data, suggesting such models might not generalize to other subjects. However, prediction of synthetic data was continuously improved by adding synthetic data from other sampling distributions for subject backgrounds. This illustrates synthetic data can improve generalizability of complex ML and DL models while reducing overfitting, although many thousands of perturbation experiments may be required.

Panels (b) and (c) demonstrate that while synthetic samples may degrade performance of simple models like Lasso on observed subjects, this effect is relatively small with complex models, which ultimately can make better predictions. While predictions of observed data using models trained only on synthetic data was not particularly strong without some observed backgrounds, these results illustrate some general features may be learned from synthetic data, which can enable pre-trained models for few-shot learning on patient samples.

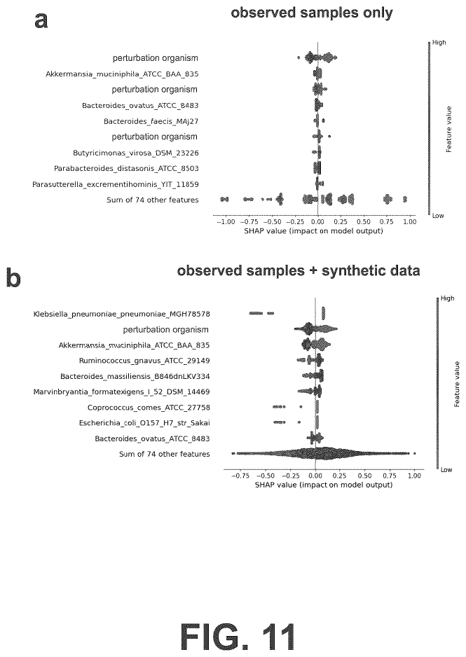

The explainable AI module 138 was evaluated to demonstrate the utility of the present disclosure to identify causal microbiome targets, which is illustrated by model feature exploration (shown here by SHAP plots). FIG. 11 shows top ranking feature effects of trained models (microbial taxa influence on model outcomes) as assessed by SHAP. The x-axis shows whether feature impact is positive or negative, while each point is an observation with relatively high (red) or low (blue) feature values (taxa counts). Taxa with positive impacts on outcome are mostly red to the right of the 0 line on the x-axis; those with negative impacts are mostly red to the left of the 0 impact line (negative). Panel (a) shows results from training models using only observed samples (corresponding to models in FIG. 9), while panel (b) shows scores using the full complement of synthetic data (corresponding to models in FIG. 10).

Closer investigation of the SHAP plots in FIG. 11 illustrates that synthetic data may not only produce more robust and generalizable models, but further contribute better discrimination of effects by reducing overfitting in microbiome studies where n<<p-assuming that such synthetic samples may have their outcomes measured as the system was able to using the microbiome digital twin platform 300.

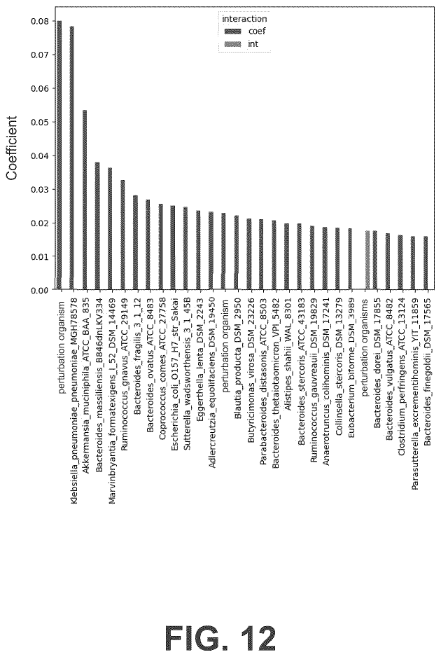

Another explainable approach used in the explainable AI module 138 is the use of coefficient plots for EBM models that identifies model features that demonstrate strong effects on the outcome. For example, although in SHAP plots in FIG. 11 some of the organisms involved in perturbation of the microbiomes appeared lower ranked once trained on synthetic data, coefficient plots in FIG. 12, show an emergence of a significant interaction of some of those organisms which was not present in models trained only on observed samples, which further illustrates that synthetic data may further contribute better discrimination of effects by reducing overfitting.

It should be appreciated that while the above example focuses on target identification at the microbial level (i.e. identification of positive and negative microbial taxa), the same process could be applied to any other component of the microbiome. As illustrative and non-limiting examples, microbes, microbe-derived metabolites, microbial genes, metabolic pathways, or any combination thereof could be identified as potential targets.

Results from modeling with synthetic samples plainly illustrate the potential for machine learning to overfit models to a small number of patient backgrounds, yet also provide a path to overcome this limitation. The microbiome digital twin platform 300 uniquely enables observation of the outcomes of synthetic backgrounds in experimental manipulations, in the same manner as patient backgrounds.

The results presented here further suggest that synthetic data may be viewed as a method by which to sample unseen perturbations (by varying degrees of randomness) or to better understand effects of components of the microbiome that do not universally appear in observed samples such as rare organisms.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods.

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers.

One or more aspects of the disclosure may be embodied in computer-usable data and computer-executable instructions, such as in one or more program modules, executed by one or more computers (including monitoring modules), or other devices. Generally, program modules include routines, programs, objects, components, data structures, and so on, that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGAs, and the like.

Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well of any individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well of any individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The previously described versions of the disclosed subject matter have many advantages that were either described or would be apparent to a person of ordinary skill. Even so, these advantages or features are not required in all versions of the disclosed apparatus, systems, or methods.

Additionally, this written description makes reference to particular features. It is to be understood that the disclosure in this specification includes all possible combinations of those particular features. Where a particular feature is disclosed in the context of a particular aspect or example, that feature can also be used, to the extent possible, in the context of other aspects and examples.

Also, when reference is made in this application to a method having two or more defined steps or operations, the defined steps or operations can be carried out in any order or simultaneously, unless the context excludes those possibilities.

Although specific examples of the invention have been illustrated and described for purposes of illustration, it will be understood that various modifications may be made without departing from the spirit and scope of the invention.

What is claimed is:

1. A method for determining one or more causal microbiome features impacting a medical condition, comprising:
receiving, from a plurality of patients with the medical condition, a plurality of microbiome samples;
generating a set of perturbed microbiomes from the plurality of microbiome samples;
evaluating a set of objective functions at least in part by processing the set of perturbed microbiomes, wherein an objective function of the set of objective functions relates to an association of a microbiome with the medical condition; and
identifying, by processing the set of objective functions and the set of perturbed microbiomes, the one or more causal microbiome features impacting the medical condition.

2. The method of claim 1, wherein evaluating the set of objective functions and identifying the one or more causal microbiome features are performed using at least one machine learning model.

3. The method of claim 2, wherein the evaluating the set of objective functions is performed using a machine learning model of the at least one machine learning model and the identifying the one or more causal microbiome features is performed using a post-hoc explainability process applied to the machine learning model.

4. The method of claim 3, wherein the post-hoc explainability process comprises Local Interpretable Model-agnostic Explanations (LIME) or SHapley Additive explanations (SHAP).

5. The method of claim 2, wherein evaluating the set of objective functions is performed using an inherently explainable machine learning model and identifying the one or more causal microbiome features is performed using feature importance or model-specific explainability methods.

6. The method of claim 1, wherein a microbiome sample of the plurality of microbiome samples is a digitized sample collected from a patient of the plurality of patients or a synthetically generated sample.

7. The method of claim 1, wherein a microbiome sample of the plurality of microbiome samples comprises a plurality of microbes.

8. The method of claim 7, wherein a microbe of the plurality of microbes is a pathogen.

9. The method of claim 8, wherein the microbe is a bacterium.

10. The method of claim 2, further comprising: providing the one or more causal microbiome features as input to the at least one machine learning model.

11. The method of claim 1, wherein the medical condition is associated with a gut microbiome, a skin microbiome, a vaginal microbiome, or an oral microbiome of a patient.

12. The method of claim 1, wherein the medical condition is a disease.

13. The method of claim 12, wherein the disease is Crohn's disease, hepatic encephalopathy, ulcerative colitis, Type 2 diabetes, irritable bowel syndrome (IBS), Alzheimer's disease, or Parkinson's disease.

14. The method of claim 1, wherein the medical condition is an allergy or acne.

15. The method of claim 6, wherein the synthetically generated sample is generated using a statistical method or a generative artificial intelligence method.

16. The method of claim 15, wherein the statistical method is Gaussian Copula.

17. The method of claim 15, wherein the generative artificial intelligence method is a tabular variational autoencoder or a generative adversarial network (GAN).

18. The method of claim 1, wherein generating a perturbed microbiome of the set of perturbed microbiomes comprises a modifying a metabolic landscape of a microbiome sample of the plurality of microbiome samples.

19. The method of claim 18, wherein the modifying the metabolic landscape is performed in silico, in vitro, or in vivo.

20. The method of claim 18, wherein the modifying the metabolic landscape relates to a modification of a microbiome composition or function.

21. The method of claim 20, wherein the modification comprises introducing or removing one or more microbial strains, changing a concentration of metabolites, modifying an activity of a microbial gene, or any combination thereof.

22. The method of claim 1, wherein a feature of the one or more causal microbiome features is a presence, an abundance, an activity, a concentration, or any combination thereof of a target microbiome component.

23. The method of claim 22, wherein the target microbiome component comprises a microbe, a microbe-derived metabolite, a microbial gene, a metabolic pathway, or a combination thereof.

24. The method of claim 3, wherein the machine learning model comprises least absolute shrinkage and selection operator (Lasso), random forests, or a neural network.

25. The method of claim 5, wherein the inherently explainable machine learning model comprises an explainable boosting machine (EBM).

26. The method of claim 15, wherein generating the synthetically generated sample comprises selecting a subset of nearest neighbor microbiome samples.

27. A system for determining one or more causal microbiome features impacting a medical condition, comprising one or more computer processors individually or collectively programmed to:
receive, from a plurality of patients with the medical condition, a plurality of microbiome samples;
generate a set of perturbed microbiomes from the plurality of microbiome samples;
evaluate a set of objective functions at least in part by processing the set of perturbed microbiomes, wherein an objective function of the set of objective functions relates to an association of a microbiome with the medical condition; and
identify, by processing the set of objective functions and the set of perturbed microbiomes, the one or more causal microbiome features impacting the medical condition.

28. A non-transitory computer-readable medium comprising machine-executable code that, upon execution by one or more computer processors, implements a method for determining one or more causal microbiome features impacting a medical condition, comprising:
receiving, from a plurality of patients with the medical condition, a plurality of microbiome samples;
generating a set of perturbed microbiomes from the plurality of microbiome samples;
evaluating a set of objective functions at least in part by processing the set of perturbed microbiomes, wherein an objective function of the set of objective functions relates to an association of a microbiome with the medical condition; and
identifying, by processing the set of objective functions and the set of perturbed microbiomes, the one or more causal microbiome features impacting the medical condition.
